(19) United States
(12) Patent Application Publication
(10) Pub. No.: US 2018/0220892 A1
(43) Pub. Date: Aug. 9, 2018

(54) ASSESSMENT OF MICROVASCULAR DYSFUNCTION WITH SPECTRAL IMAGING

(71) Applicants: Corning Incorporated, Corning, NY (US); University of Rochester, Rochester, NY (US)

(72) Inventors: William Spencer Klubben, III, Corning, NY (US); Anthony Pietropaoli, Pittsford, NY (US); Paul Michael Then, Victor, NY (US)

(21) Appl. No.: 15/889,771

(22) Filed: Feb. 6, 2018

Related U.S. Application Data
(60) Provisional application No. 62/456,765, filed on Feb. 9, 2017.
(60) Provisional application No. 62/546,150, filed on Aug. 16, 2017.

(51) Int. Cl. A61B 5/00; A61B 1/04; A61B 90/20; A61B 5/0205; A61B 5/02

(52) U.S. Cl. CPC A61B 5/0035; A61B 5/0075; A61B 5/4866; A61B 5/7278; A61B 1/04; A61B 5/14546; A61B 5/4884; A61B 5/0205; A61B 5/02007; A61B 2505/05; A61B 90/20

(57) ABSTRACT

A method for quantifying microvascular function in a patient includes stabilizing a test portion of the patent to analyze. A microvascular blood flow parameter is measured using a first spectral imaging technique. A microvascular reserve parameter is measured using a second spectral imaging technique. A tissue respiration parameter is measured using a third spectral imaging technique. A microvascular permeability parameter is measured using a fourth spectral imaging technique. The method further includes processing the microvascular blood flow parameter, the microvascular reserve parameter, the tissue respiration parameter, and the microvascular permeability parameter together using a processor configured to generate a summary microvascular parameter corresponding to the microvascular function in the patient.

This application claims the benefit of priority under 35 U.S.C. § 119 of U.S. Provisional Application Ser. No. 62/456,765 filed on Feb. 9, 2017 and of Provisional Application Ser. No. 62/546,150 filed on Aug. 16, 2017, the content of which is relied upon and incorporated herein by reference in its entirety.

### TECHNICAL FIELD

The present disclosure relates generally to the spectral imaging of microvasculature. More particularly, embodiments described herein relate to quantifying microvascular dysfunction in a patient using numerous parameters.

### BACKGROUND

The efficient and accurate quantification of microvascular function (MVF) addresses clinical needs in a variety of medical specialties including surgical, diagnostic, and preventative applications. Ensuring and maintaining proper microvascular function is crucial to the health of essentially every tissue in the human body. One exemplary life-threatening condition that could be better treated or prevented by measuring a patient's microvascular function is sepsis.

Sepsis is a full-body inflammation arising from complications due to an infection. Sepsis is one of the most common and expensive reasons for hospitalization in the United States where 28-50% of patients who become septic die. Since sepsis disproportionately affects people over 65, effective treatment is a growing and substantial clinical need. One reason why sepsis is such a large problem is because it is difficult to diagnose, quantify, and monitor. The primary cause of morbidity and mortality in sepsis patients is organ failure. The vascular system circulates the chemicals that trigger inflammation and these chemicals are predominantly activated at the location of chemical transport—the microvasculature. Thus, microvascular dysfunction triggered by the inflammatory response of sepsis greatly contributes to organ failure. By capturing pertinent information related to a patient's microvascular health, clinicians may be better suited to more efficiently quantify and monitor sepsis in patients.

The current methods used for quantifying microvascular function in a patient typically include a variety of techniques such as, for example, pulse oximetry capillary refill rate, buccal mucosa microvascular imaging, and forearm laser Doppler flowmetry. Although these various techniques are suited for obtaining limited data, the currently used methods generally do not offer a holistic evaluation of microvascular function.

### SUMMARY

According to one embodiment, a method of quantifying microvascular function in a patient is provided. The method of quantifying microvascular function in a patient includes stabilizing a test portion of the patient to analyze, measuring a microvascular blood flow parameter of the test portion using a first spectral imaging technique, measuring a microvascular reserve parameter of the test portion using a second spectral imaging technique, measuring a tissue respiration parameter of the test portion using a third spectral imaging technique, and measuring a microvascular permeability parameter of the test portion using a fourth spectral imaging technique. The method of quantifying microvascular function in a patient further includes processing the microvascular blood flow parameter, the microvascular reserve parameter, the tissue respiration parameter, and the microvascular permeability parameter together using a processor configured to generate a summary microvascular parameter corresponding to the microvascular function in the patient.

According to another embodiment, a method of quantifying microvascular function in a patient is provided. The method of quantifying microvascular function in a patient includes measuring two or more microvascular parameters selected from the group consisting of a microvascular blood flow parameter, a microvascular reserve parameter, a tissue respiration parameter, and a microvascular permeability parameter and processing the two or more microvascular parameters using a processor configured to generate a summary microvascular parameter corresponding to the microvascular function in the patient. The two or more microvascular parameters are measured using a spectral imaging technique including microscopic spectral imaging, endoscopic spectral imaging, camera spectral imaging, or a combination thereof.

According to yet another embodiment, an instrument used to quantify microvascular function is provided. The instrument used to quantify microvascular function includes a spectral imaging device configured to measure two or more microvascular parameters selected from the group consisting of a microvascular blood flow parameter, a microvascular reserve parameter, a tissue respiration parameter, a microvascular permeability parameter, and a processor configured to generate a summary microvascular parameter from the two or more microvascular parameters.

According to yet another embodiment, a method of quantifying microvascular blood flow in a buccal mucosa or a tongue of a patient includes stabilizing a test portion of the patient to analyze, positioning a coherent light source and a spectral imaging system in a transmission geometry with respect to the test portion, and measuring a microvascular blood flow parameter of the test portion using the spectral imaging system. The test portion includes a lip, a cheek, a tongue, or a combination thereof.

The present disclosure extends to a method of quantifying microvascular function in a patient, the method comprising: stabilizing a test portion of the patient to analyze; and measuring a microvascular blood flow parameter of the test portion using a spectral imaging technique.

The present disclosure extends to a method of quantifying microvascular function in a patient, the method comprising: stabilizing a test portion of the patient to analyze; and measuring a microvascular reserve parameter of the test portion using a spectral imaging technique.

The present disclosure extends to a method of quantifying microvascular function in a patient, the method comprising: stabilizing a test portion of the patient to analyze; measuring a tissue respiration parameter of the test portion using a spectral imaging technique.

The present disclosure extends to a method of quantifying microvascular function in a patient, the method comprising: stabilizing a test portion of the patient to analyze; and measuring a microvascular permeability parameter of the test portion using a spectral imaging technique.

The present disclosure extends to a method of quantifying microvascular function in a patient, the method comprising: stabilizing a test portion of the patient to analyze; measuring a microvascular blood flow parameter of the test portion using a first spectral imaging technique; measuring a microvascular reserve parameter of the test portion using a second spectral imaging technique; measuring a tissue respiration parameter of the test portion using a third spectral imaging technique; measuring a microvascular permeability parameter of the test portion using a fourth spectral imaging technique; and processing the microvascular blood flow parameter, the microvascular reserve parameter, the tissue respiration parameter, and the microvascular permeability parameter together using a processor configured to generate a summary microvascular parameter corresponding to the microvascular function in the patient.

The present disclosure extends to a method of quantifying microvascular function in a patient, the method comprising: measuring two or more microvascular parameters selected from the group consisting of a microvascular blood flow parameter, a microvascular reserve parameter, a tissue respiration parameter, and a microvascular permeability parameter; and processing the two or more microvascular parameters using a controller configured to generate a summary microvascular parameter corresponding to the microvascular function in the patient; wherein the two or more microvascular parameters are measured using a spectral imaging technique comprising microscopic spectral imaging, endoscopic spectral imaging, camera spectral imaging, or a combination thereof.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

FIG. 1 is a schematic flow diagram illustrating a method for quantifying microvascular function in a patient according to one embodiment;

FIG. 2 is a schematic representation of a hyperspectral camera that may be used in the method;

FIG. 3 is a schematic block/flow diagram illustrating an interface between a user, test portion, and device according to one embodiment;

FIG. 4 is an exemplary representation of microvascular circulation;

FIG. 5 is a series of images taken using a hyperspectral camera with different spectral bands in reflectance mode according to one example;

FIG. 6 is a perspective view of a mouth to be imaged using a hyperspectral camera in transmission mode according to one embodiment; and

FIG. 7 is a schematic flow diagram illustrating a method for quantifying microvascular function in a patient according to another embodiment.

### DETAILED DESCRIPTION

The methods and respective device herein uses spectral imaging to image a patient's microvasculature to obtain information regarding the local and overall microvascular health of the patient. As is understood in the art, the overall microvasculature health of a patient as described herein is linked to the prognosis of sepsis, however there may be many other additional clinical applications for this technology for example in surgical, diagnostic, and preventative uses. In some embodiments, the use of this method and device is to provide clinicians with critical information regarding the health of a patient's microvasculature to better diagnose, treat, manage, and detect issues with current, suspected, or past sepsis. In some embodiments, the quantification of the microvascular function in a patient is measured as a summary microvascular parameter derived from using a combination of two or more metrics: microvascular blood flow, microvascular reserve, tissue respiration, and microvascular permeability. Current analytical techniques analyze and provide data for only one of these four metrics. The methods associated with processing the images obtained using spectral techniques are the core principles disclosed herein. While modifications to a pulse oximetry device can illicit information regarding microvascular reserve and a sidestream dark field imaging device may be able to provide information regarding permeability, neither of these currently available devices combine the microvascular blood flow, microvascular reserve, tissue respiration, and microvascular permeability metrics to provide a big picture measure of a patient's overall microvascular health.

By using spectral imaging technology to produce images of the microvasculature, these images will prove to be relevant and are likely to find clinical acceptance because the images and related data will be more familiar than purely spectroscopic data. Methods known in the prior art generally are not able to yield quantifiable data for multiple metrics of microvascular dysfunction as disclosed herein.

Reference will now be made in detail to the present preferred embodiments, examples of which are illustrated in the accompanying drawings. Whenever possible, the same reference numerals will be used throughout the drawings to refer to the same or like parts.

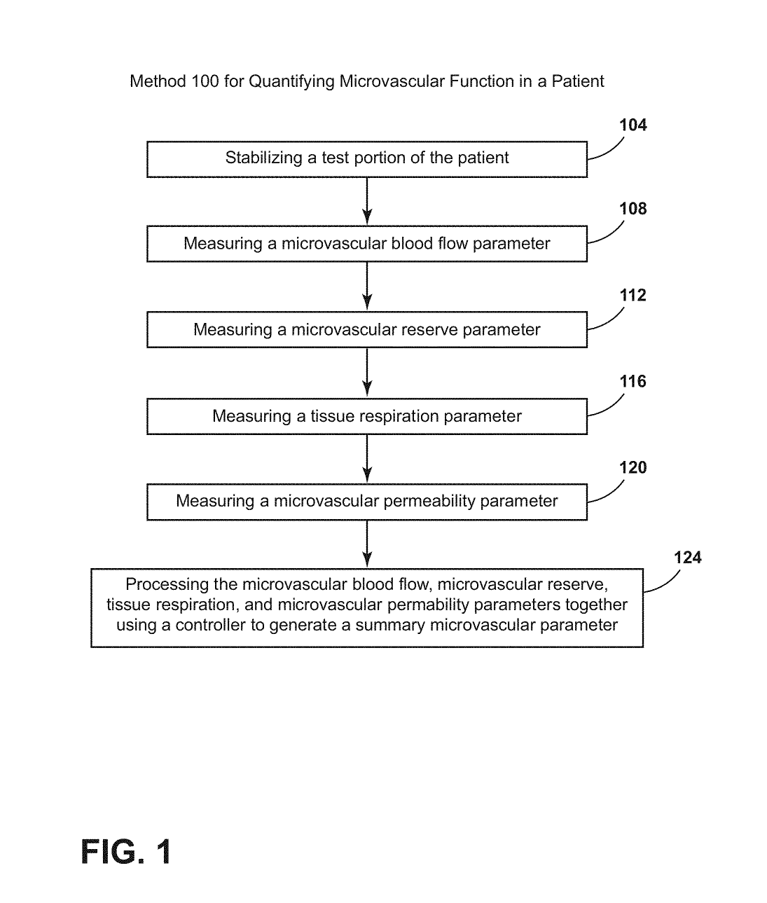

Referring now to FIG. 1, according to a first embodiment, a method 100 is disclosed for quantifying microvascular function in a patient, such as a human being. The method 100 of quantifying microvascular function in a patient includes stabilizing a test portion of the patient to analyze at step 104. A microvascular blood flow parameter of the test portion is measured using a first spectral imaging technique at step 108. A microvascular reserve parameter of the test portion is measured using a second spectral imaging technique at step 112. A tissue respiration parameter of the test portion is measured using a third spectral imaging technique at step 116. A microvascular permeability parameter of the test portion is measured using a fourth spectral imaging technique at step 120. The method 100 of quantifying microvascular function in a patient further includes processing the microvascular blood flow parameter, the microvascular reserve parameter, the tissue respiration parameter, and the microvascular permeability parameter together using a controller configured to generate a summary microvascular parameter corresponding to the microvascular function in the patient at step 124.

Each of the first, second, third, and fourth spectral imaging techniques may be performed bedside and may use one or more hyperspectral imaging cameras in combination with various wavelength bands to image and quantify the microvascular blood flow, the microvascular reserve, the tissue respiration, and the microvascular permeability parameters, respectively. The hyperspectral camera may include a wavelength-dispersing element and a detection element. The wavelength-dispersing element receives light and separates or disperses light according to wavelength. The wavelength-dispersing element may include optics such as prisms, lenses, and mirrors. The wavelength-dispersing element may be a spectrometer. The spectrometer may be an Offner spectrometer. An Offner spectrometer is a particularly compact spectrometer that enables miniaturization of the present hyperspectral imaging system. An example of an Offner spectrometer is described in U.S. Pat. No. 7,697,137, the disclosure of which is hereby incorporated by reference in its entirety herein. The wavelength-dispersing element may direct light to the detection element. The detection element may detect the wavelength, intensity, polarization or other characteristic of the light dispersed by the wavelength-dispersing element. The detection element may be a photodetector, a CCD device, a diode array, a focal plane array, a CMOS device, or other types of image detector known in the art for sensing electromagnetic radiation reflected over the wavelength range associated with physical objects in real-world scenes.

Each of the microvascular blood flow, the microvascular reserve, the tissue respiration, and the microvascular permeability parameters may be measured by detecting one or more compounds in the blood, tissue, cells, extracellular fluid, and/or arterial/capillary walls. For example, in some embodiments, the detected compounds may include carbon dioxide (CO₂), oxygen (O₂), hemoglobin, oxyhemoglobin, deoxyhemoglobin, carboxyhemoglobin, methemoglobin, nitric oxide, and/or other compounds known in the art that are found in the body that may be measured spectroscopically. In addition, each of the microvascular blood flow, the microvascular reserve, the tissue respiration, and the microvascular permeability parameters may be measured by monitoring the difference (Δ) or rate of change (kinetics) for one or more of the detectable compounds mentioned above using a combination of occlusion and reperfusion. By occluding and then reperfusing the vasculature, the change and/or kinetics of the detectable compounds measured, as related to the respective vascular parameter, can provide various amounts of useful information to the caregiver.

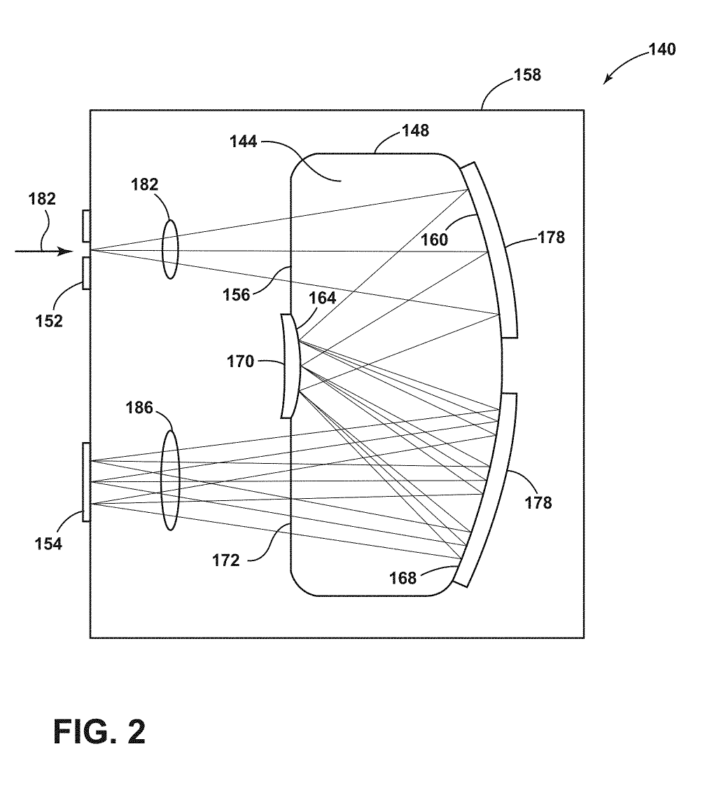

FIG. 2 illustrates a hyperspectral camera that includes a monolithic Offner spectrometer and a detector. The hyperspectral camera 140 incorporates a monolithic Offner spectrometer 148 within an optical housing 158. Hyperspectral camera 140 includes a slit 152 and a detector 154 attached to the optical housing 158. In the configuration shown, the monolithic Offner spectrometer 148 is a one-to-one optical relay made from a single piece of transmissive material 144 including an entrance surface 156, a first mirror 160 (formed when a reflective coating 178 is applied as shown to the surface of transmissive material 144), a diffraction grating 164 (formed when a reflective coating 178 is applied as shown to the surface of transmissive material 144), a second mirror 168 (formed when a reflective coating 178 is applied as shown to the surface of transmissive material 144) and an exit surface 172.

The hyperspectral camera 140 operates to produce images of a remote object (not shown) over a contiguous range of narrow spectral bands when the slit 152 receives a beam 182 from the remote object and directs the beam 182 to the monolithic Offner spectrometer 148. Monolithic Offner spectrometer 148 diffracts the beam 182 and forwards the diffracted beam 186 to the detector 154. In particular, the slit 152 directs the beam 182 to the entrance surface 156. First mirror 160 receives the beam 182 transmitted through the entrance surface 156 and reflects the beam 182 towards the diffraction grating 164. The diffraction grating 164 receives the beam 182 and diffracts and reflects the diffracted beam 186 to the second mirror 168. The second mirror 168 receives the diffracted beam 186 and reflects the diffracted beam 186 to the exit surface 172. The detector 154 processes the diffracted beam 186 received from exit surface 172.

Still referring to FIG. 2, transmissive material 144 is selected to have high transparency over the range of wavelengths acquired from the scene during imaging. Wavelengths of interest may include near infrared wavelengths, visible wavelengths, and/or ultraviolet wavelengths. Materials suitable for transmissive material 144 include plastics, dielectrics, and gases (e.g. air, nitrogen, argon, etc.). When gases are employed, first mirror 160, second mirror 168, and reflective coating 178 are affixed to optical housing 158 through posts or other mounts.

Detector 154 is selected to have a wavelength (color) sensitivity based on the type of transmissive material 144 used to make the monolithic Offner spectrometer 148. For instance, if the monolithic Offner spectrometer 148 were made from a plastic (e.g., polymethylmethacrylate (PMMA), polystyrene, polycarbonate) then the diffracted wavelength range would be primarily in the visible and the detector 154 could be a complementary metal-oxide-semiconductor (CMOS) video camera 154. If the monolithic Offner spectrometer 148 were made from an infrared transmitting material, then the detector 154 would be an IR detector, such as one based on mercury cadmium telluride (HgCdTe), indium antimonite (InSb) or lead sulphide (PbS).

Hyperspectral camera 140 may further include additional optics to receive or direct a light beam 182 and/or a diffracted light beam 186 to or from different directions to permit flexible positioning of slit 152 and/or detector 154 with respect to optical housing 158. The hyperspectral imaging system may also include a battery module (not shown). The battery module may include a rechargeable battery and may be removably coupled to the hyperspectral camera, a mobile display device, or other module of the hyperspectral imaging system. Battery power may also be provided by a battery contained within the mobile display device. The hyperspectral imaging system may also be adapted to receive power from an external battery.

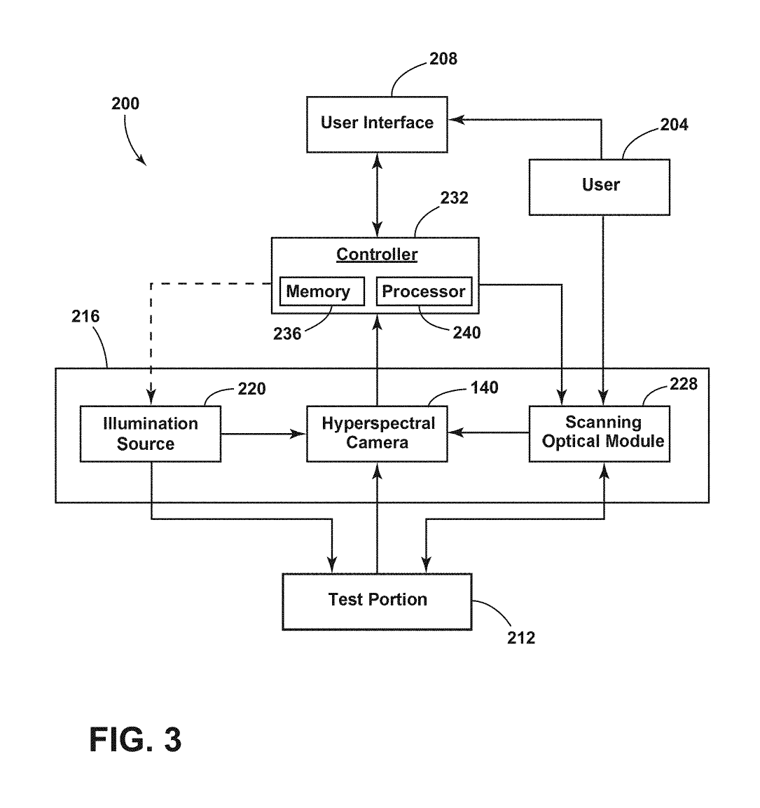

Referring now to FIG. 3, a hyperspectral imaging system 200 includes an illumination source 220 that generates light that contacts the test portion 212 and is then transmitted or directed to the hyperspectral camera 140 and/or a scanning optical module 228. The hyperspectral camera 140 may include a controller 232 to process image data acquired from the patient. The image data may include spectral data, wavelength data, polarization data, intensity data, and/or positional data. The controller 232 may receive image data through a processor 240 from the test portion 212 or detection element and transform or otherwise manipulate the image data into the summary microvascular parameter specified by a user 204. The user 204 may use an user interface 208 to select various specifications for the various hyperspectral camera 140 and scanning optical module 228 such as wavelengths used by the first, second, third, and fourth spectral imaging techniques. Data processing may include conversion of image data to any of several visual forms known in the art and may include coloring, shading, or other visual effects intended to represent position, depth, composition, motion, or other features of objects in the scene. In some embodiments, the data processing performed by the processor 240 may include conversion of the image data to the respective microvascular blood flow parameter, the microvascular reserve parameter, the tissue respiration parameter, or the microvascular permeability parameter.

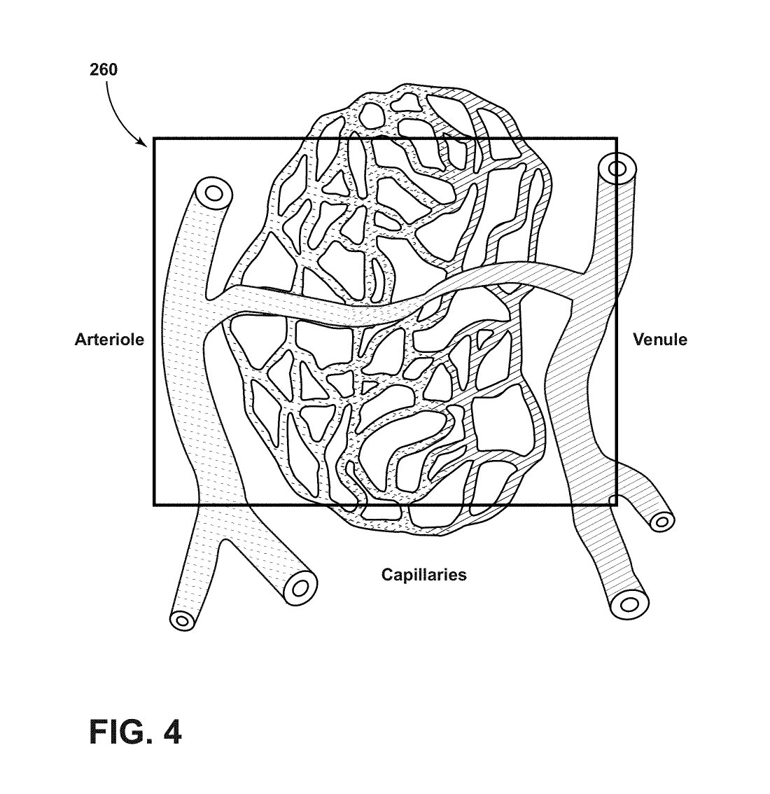

Specifically, the image data may be used to map and/or measure the concentration of components of interest, which may then be used to calculate the desired parameter. The microvascular blood flow parameter is a measure of the blood flow through the vessels or tissues. The microvascular blood flow parameter may be calculated by measuring a change in spectroscopic intensity speckle change, as described in greater detail below. The microvascular reserve parameter is a computed quantity that is an index of relative microcirculatory perfusion reserve, which is calculated by using the ratio of the average baseline perfusion (q35) to the perfusion achievable at 45° (q45) according to the equation MVR (%)=[1−(q35/q45)]*100. Perfusion is the passage of fluid through the circulatory system to an organ or tissue, and specifically, the delivery of blood to a capillary bed. The microvascular reserve parameter may be calculated based on an observed rate of response to an ischemic stimuli, as described below. The tissue respiration parameter is a measure of the interchange of gases that occurs between the blood and the tissues. The tissue respiration parameter may be calculated based on the concentrations of oxygenated hemoglobin and deoxygenated hemoglobin, determined based on the image data, as described below with respect to FIG. 4. The microvascular permeability parameter is a measure of the capacity of a blood vessel wall to allow for the flow of small molecules (including drugs, nutrients, water, ions, and the like” or even whole cells (such as lymphocytes) in and out of the vessel. The microvascular permeability parameter may be calculated based on the mapping of water movement following an occlusion.

In additional embodiments, the data processing performed by the controller 232 and/or processor 240 may include conversion of the respective microvascular blood flow parameter, the microvascular reserve parameter, the tissue respiration parameter, and/or the microvascular permeability parameter to generate the summary microvascular parameter and/or a summary microvascular image.

Data received and/or processed by the hyperspectral camera may be transferred to a mobile display device for further processing and/or display. The data transfer may occur through a data interface, such as a data link or USB connection. The hyperspectral camera 140 may also include a memory 236. The memory 236 may be used to store image data. The image data may be unprocessed or processed image data. Image data stored in the hyperspectral camera may be downloaded to an external computer for processing. Image data stored in the hyperspectral camera may be processed offline.

The hyperspectral imaging system 200 may include the scanning optical module 228. The scanning optical module 228 may include moveable optics for scanning a scene. The moveable optics may acquire image data from a slice of a scene and may be systematically repositioned or reconfigured to continuously sample a scene in a slice-by-slice fashion. Slice image data acquired by the scanning optical module may be directed to the hyperspectral camera 140 for acquisition and processing. The scanning optical module 228 may include rotatable optical elements, such as a rotatable mirror or lens. The scanning optical module 228 may be removably coupled to the hyperspectral camera 140, a mobile display device, or a rechargeable battery module.

Still referring to FIG. 3, the illumination source 220, hyperspectral camera 140, and the scanning optical module 228 together represent at least one spectral imaging technique 216. In some embodiments, two, three, four, or more spectral imaging techniques and their associated components may be coupled to the processor 240 to generate the summary microvascular parameter and/or the summary microvascular image.

The method of both quantifying the microvascular blood flow parameter, the microvascular reserve parameter, the tissue respiration parameter, and the microvascular permeability parameter together using the hyperspectral imaging system 200 and combining these parameters using the processor 240 to provide/generate the summary microvascular parameter and/or summary microvascular image can be performed using a variety of different techniques. In some embodiments, the summary microvascular parameter may be one or more values that correspond to the general or overall microvascular health of the patient. In other embodiments, the summary microvascular image may be an image that corresponds to the general or overall microvascular health of the patient, for example, a heat map or other averaged image taking the respective microvascular blood flow, the microvascular reserve, the tissue respiration, and/or the microvascular permeability parameters or data into the calculation of the image.

The microvascular blood flow parameter can be measured using the first spectral imaging technique. In some embodiments, the first spectral imaging technique may be a line scan spectroscopic speckle technique. The line scan spectroscopic speckle technique is performed by stabilizing a test portion of the patient to analyze and then using the hyperspectral camera 140 to image and capture data for the test portion. In some embodiments, a full spectral scan of the test portion may be used. From the data captured from the image using the hyperspectral camera 140, a column in the image is selected either automatically or manually by the user 204 where the selected column contains a vessel of interest. Additional line scans at the selected location may be performed while keeping the test portion stabilized in order to measure and compute the blood flow at the selected group of pixels by observing a change in the spectroscopic intensity speckle change. The microvascular blood flow parameter may be measured using a wavelength between about 400 nm and about 3000 nm, about 400 nm and about 1500 nm, about 400 nm and about 800 nm, or about 530 nm and about 580 nm. Both diffuse correlation spectroscopy (DCS) and diffuse optical spectroscopy (DOS) monitor speckle change to inspect the arterial occlusion and vascular occlusion responses to establish the microvascular blood flow parameter. In some embodiments, the microvascular blood flow is quantified using a line scan spectroscopic speckle technique by scanning an entire region of the test portion and selecting a column in the image which contains a vessel of interest and conducting a line scan at that location while keeping the tissue stabilized and computing the flow at the selected group of pixels by observing a change in the spectroscopic intensity speckle change. In other embodiments, the first spectral imaging technique may be a spectroscopic technique comprising diffuse correlation spectroscopy, diffuse optical spectroscopy, or a combination thereof to detect arterial occlusion and vascular occlusion responses to calculate DCS/DOS flow values.

The microvascular reserve parameter can be measured using the second spectral imaging technique. In some embodiments, the second spectral imaging technique may observe a capillary response to an ischemic stimulus where the blood supply is at least temporarily changed or shut off. By spectrographically monitoring the rate of response to the ischemic stimuli, a ratio of hemoglobin present before and after the occlusion can be determined. By releasing the ischemic stimulus during a spectral scan, the scanned test portion can be monitored during and after the release of the ischemic stimulus. The microvascular reserve parameter may be measured using a wavelength between about 400 nm and about 1500 nm, about 400 nm and about 800 nm, about 530 nm and about 580 nm, or a wavelength between about 440 nm and about 460 nm.

The tissue respiration parameter can be measured using the third spectral imaging technique. In some embodiments, the third spectral imaging technique may use spectral classification such as pulse oximetry to image tissue respiration molecules such as hemoglobin (Hb) and oxyhemoglobin (HbO₂). Referring to FIG. 4, a representative perfusion area 260 is shown where tissue respiration can be measured where tissue oxygenation is mapped as a function of location along a vessel. In some embodiments, sepsis, by its nature, is heterogeneous throughout the body, especially its effect on the microvasculature. Thus, being able to image and quantify a large area of interest would be advantageous and potentially differentiating from other techniques. The tissue respiration parameter may be measured using a wavelength between about 400 nm and about 1500 nm, about 400 nm and about 800 nm, about 530 nm to about 580 nm, or a wavelength between about 440 nm and about 460 nm.

The microvascular permeability parameter can be measured using the fourth spectral imaging technique. In some embodiments, the fourth spectral imaging technique may image how water (H₂O) permeates through a plurality of vascular walls of the microvasculature. By spectrographically monitoring water movement after the application of an occlusion, one may gauge how fast it takes for water levels to return to normal. According to the enhanced permeation and retention (EPR) effect, if the microvasculature is leaky it will take longer for water to leave the occluded area. The microvascular permeability parameter may be measured using a wavelength between about 800 nm and about 2 m. The hyperspectral imaging may also monitor wavelength bands of about 820 nm and about 730 nm. Additional strong bands may be monitored via hyperspectral imaging at 2900 nm, 1950 nm, and 1450 nm; medium bands of about 1200 nm and about 900 nm; and weak bands of about 820 nm and 730 nm.

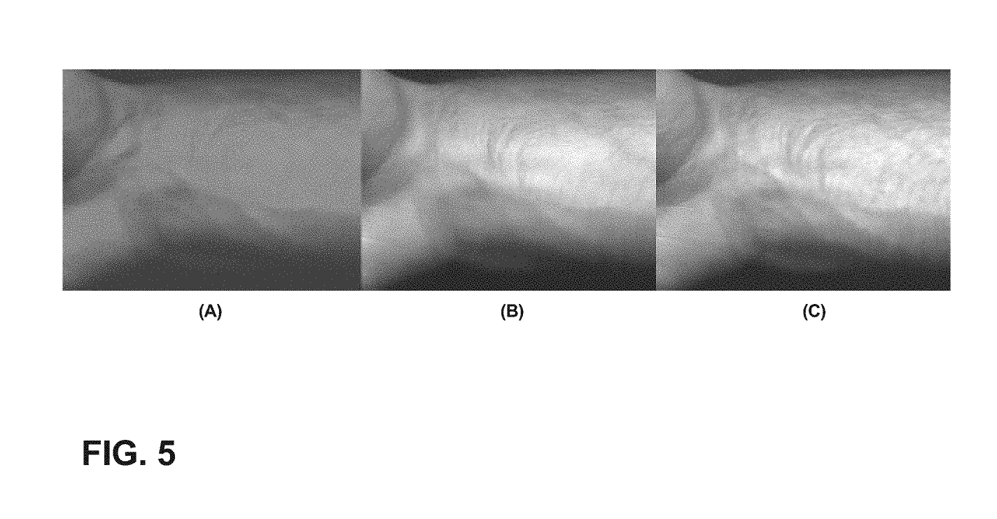

Referring to FIG. 5, a large vessel in an occluded forearm of a patient is shown using three different spectral bonds (A-C). The A image uses white light, the B image uses VNIR (visible—near-infrared) light, and the C image also uses VNIR light. As shown on the occluded forearm, a hyperspectral imaging system 200 (FIG. 3) could be used in reflectance mode to generate one of more of the microvascular blood flow parameter, the microvascular reserve parameter, the tissue respiration parameter, and the microvascular permeability parameter.

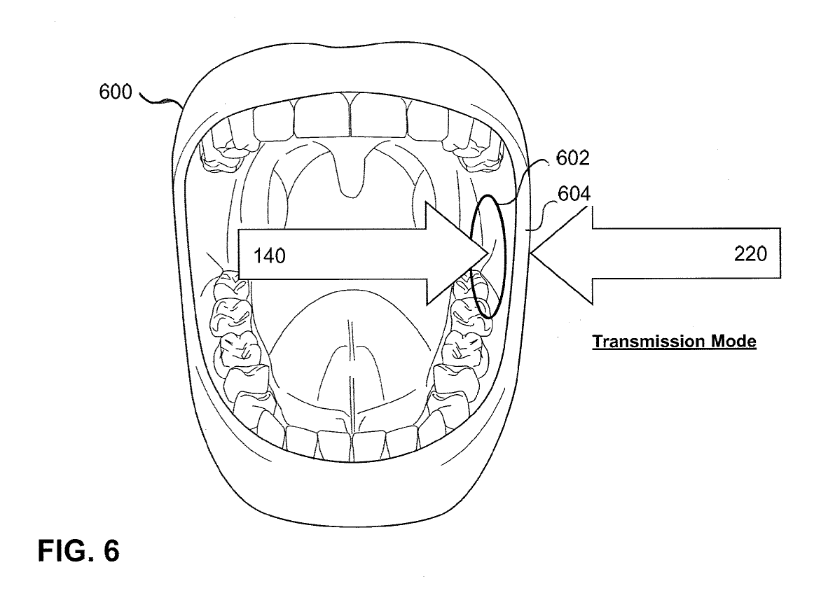

Referring to FIG. 6, a buccal imaging embodiment is shown where transmittance could be measured by the hyperspectral imaging system 200 as shown in FIG. 3. In some embodiments, a hyperspectral imaging system 200 is employed, although it is contemplated that a multispectral imaging system may be utilized in other embodiments. Accordingly, any suitable imaging system may be selected provided it is capable of detecting the particular compounds to be imaged over the field of view, including, but not limited to, H₂O, oxygenated hemoglobin, deoxygenated hemoglobin, or the like. The image of an open mouth 600 of a patient depicts the general location to be imaged 602 (black ring) and the locations of the illumination source 220 and the hyperspectral camera 140. Although in the embodiment depicted in FIG. 6, a hyperspectral camera 140 is shown, it is contemplated that in other embodiments, a scanning optical module 228 or other imaging system detector may be used in addition to, or as an alternative to, the hyperspectral camera 140.

In particular, in the embodiment depicted in FIG. 6, the hyperspectral camera 140 is positioned within the open mouth 600 of the patient, while the illumination source 220 is positioned outside of the mouth 600 with the location to be imaged 602 between the illumination source 220 and the hyperspectral camera 140, such that the illumination source 220 and the hyperspectral camera 140 are positioned in a transmission geometry with respect to the location to be imaged 602. As used herein, “transmission geometry” refers to an arrangement in which the location to be imaged 602 is oriented 180° from the hyperspectral camera 140 and the illumination source 220, with the location to be imaged 602 positioned between the hyperspectral camera 140 and the illumination source 220. However, in some embodiments, the illumination source 220 may be positioned within the open mouth 600 while the hyperspectral camera 140 is positioned outside of the mouth 600 with the location to be imaged 602 between the illumination source 220 and the hyperspectral camera 140. Although various embodiments herein contemplate that the illumination source 220 and the hyperspectral camera 140 are positioned in a transmission geometry, it is contemplated that a reflection geometry may be utilized in some particular embodiments. As used herein, “reflection geometry” refers to an arrangement in which the location to be imaged 602 is positioned parallel, or some angle between 0 and 180 degrees to the hyperspectral camera 140 and the illumination source 220 directs light onto the location to be imaged 602 at a predetermined angle. In a reflection geometry, the hyperspectral camera 140 and the illumination source 220 are on the same side of the location to be imaged 602. However, it is believed that the use of a transmission geometry may achieve more significant depths of tissue penetration.

In FIG. 6, the location to be imaged 602 is a portion of the patient's cheek 604, although other areas of the mouth may be selected. For example, the location to be imaged may include a lip, a tongue, a cheek, or a combination thereof. Without being bound by theory, it is believed that measuring microvascular function using the buccal mucosa or the tongue as a location to be imaged 602, temperature fluctuations may be reduced as compared to using another location that is covered by skin, such as those locations described in greater detail herein. Additionally, it is believed that the microvasculature may be more readily exposed in the tongue because of the lack of skin. Accordingly, it is believed that such embodiments may result in an increase in the signal to noise ratio, and a decrease in variability of the measurements. However, it is contemplated that other locations may be employed, including but not limited to, an earlobe, finger, toe, or penis, breast, or section of skin folded over on itself.

The illumination source 220 may be a coherent light source, such as a laser beam, or it may be an incoherent light source, such as an ordinary light source, including but not limited to filament, fluorescent tube light or Light Emitting Diode (LED) sources. In some embodiments, the illumination source 220 to be used is selected based at least in part on the type of spectroscopy to be employed. In one particular embodiment, the illumination source 220 is a coherent light source and the spectral imaging system employs a hyperspectral imaging technique, a line scan spectroscopic speckle technique, or a diffuse optical spectroscopic technique, as described in greater detail herein. The illumination source 220 may use a wavelength of from about 400 nm to about 3000 nm or from about 400 nm to about 1500 nm. The particular wavelength of the illumination source 220 may vary depending at least in part on the parameter(s) to be measured.

In some embodiments, the vasculature of the location to be imaged 602 can be occluded by designing a clamping mechanism on both sides of the location. For example, ring forceps may be used to occlude the cheek 604 by placing one ring on the outside of the cheek 604, proximate the illumination source 200, and one ring on the inside of the cheek 604, proximate the hyperspectral camera 140. To occlude the vasculature, the forceps may be closed to apply a pressure the cheek 604 via the rings.

In practice, the illumination source 200 transmits light through the location to be imaged 602 and the hyperspectral camera 140 images and captures data for the test portion, as described in greater detail above and below. In various embodiments, the hyperspectral camera 140 may obtain images and data corresponding to the microvascular blood flow of the patient within the location to be imaged 602. However, it is contemplated that in other embodiments, other metrics, such as microvascular permeability, tissue respiration, or microvascular reserve may be quantified, as described above and below.

Image stabilization and replication may help to quantify these four metrics, thus it might be necessary to draw or stamp a repeatable figure within the field of view of the hyperspectral camera in order to ensure the images are the same. For example, when the images are aligned, one can subtract or relate the two images with a better degree of certainty. In some embodiments, the first, second, third, and fourth spectral imaging techniques each comprise microscopic spectral imaging, endoscopic spectral imaging, camera spectral imaging, or a combination thereof.

There are many possible locations on a patient to image the microvasculature: skin through an endoscope, cheek (buccal mucosa), ear, under the eyes, and any internal organ during a surgical procedure (laparoscopic or open). Additionally, this method could provide transillumination instead of a reflectance-based system. In some embodiments, the test portion comprises an arm, a leg, a neck, a head, a shoulder, a stomach, a hand, a thigh, a calf, a heel, a foot, a toe, a knee, a finger, an elbow, a chest, a neck, a penis, a breast, a face, or a combination thereof. In other embodiments, the microvascular blood flow parameter, the microvascular reserve parameter, the tissue respiration parameter, and the microvascular permeability parameter are each measured in the epidermis, dermis, hypodermis, buccal mucosa, palpebral inferior region, palpebral superior region, ear, and the outer surface of any internal organ during a surgical, laparoscopic, or endoscopic procedure.

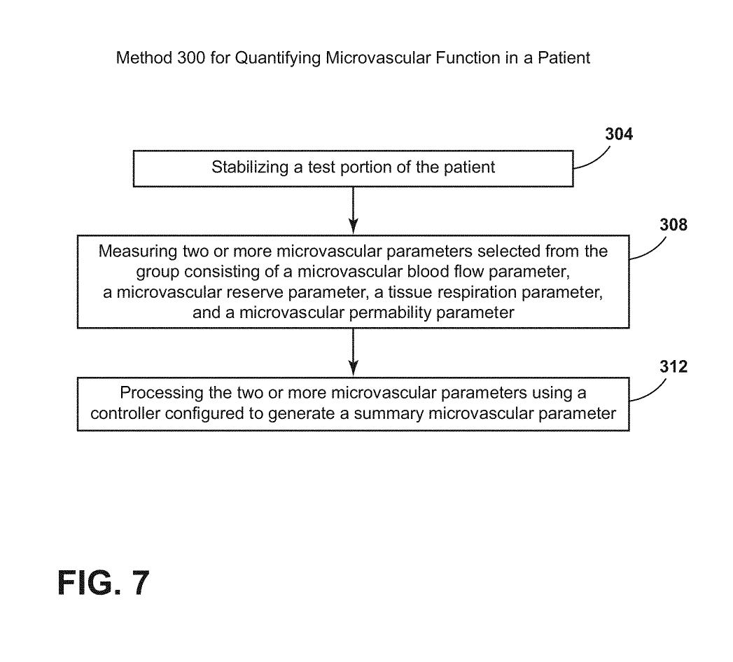

Referring now to FIG. 7, according to a second embodiment, a method 300 is disclosed for quantifying microvascular function in a patient. The method 300 of quantifying microvascular function in a patient includes stabilizing a test portion of the patient to analyze at step 304. The method 300 additionally includes measuring two or more microvascular parameters selected from the group consisting of a microvascular blood flow parameter, a microvascular reserve parameter, a tissue respiration parameter, and a microvascular permeability parameter at step 308 and processing the two or more microvascular parameters using a controller configured to generate a summary microvascular parameter corresponding to the microvascular function in the patient at step 312. The two or more microvascular parameters are measured using a spectral imaging technique comprising microscopic spectral imaging, endoscopic spectral imaging, camera spectral imaging, or a combination thereof.

It is understood that the descriptions outlining and teaching the method for quantifying microvascular function in a patient previously discussed, which can be used in any combination, apply equally well to the second embodiment of the invention, where applicable, further disclosing a method for quantifying microvascular function in a patient.

The method of both quantifying the microvascular blood flow parameter, the microvascular reserve parameter, the tissue respiration parameter, and the microvascular permeability parameter together using the hyperspectral imaging system 200 and combining these parameters using the processor 240, as shown in FIG. 3, to provide/generate the summary microvascular parameter can be performed using a variety of different techniques, as described herein.

According to a third embodiment, an instrument used to quantify microvascular function is provided. The instrument used to quantify microvascular function includes a first spectral imaging technique used to measure a microvascular blood flow parameter, a second spectral imaging technique used to measure a microvascular reserve parameter, a third spectral imaging technique used to measure a tissue respiration parameter, and a fourth spectral imaging technique used to measure a microvascular permeability parameter. The instrument used to quantify microvascular function further includes a processor configured to generate a summary microvascular parameter corresponding to the microvascular function in the patient by processing each of the microvascular blood flow, microvascular reserve, tissue respiration, and microvascular permeability parameters together.

It is understood that the descriptions outlining and teaching the methods for quantifying microvascular function in a patient previously discussed, which can be used in any combination, apply equally well to the third embodiment of the invention, where applicable, further disclosing an instrument used to quantify microvascular function.

It will be understood by one having ordinary skill in the art that construction of the described device and other components is not limited to any specific material. Other exemplary embodiments of the device disclosed herein may be formed from a wide variety of materials, unless described otherwise herein.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the device as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or description that the steps are to be limited to a specific order, it is no way intended that any particular order be inferred.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above is merely for illustrative purposes and not intended to limit the scope of the device, which is defined by the following claims as interpreted according to the principles of patent law, including the Doctrine of Equivalents.

It will be apparent to those skilled in the art that various modifications and variations can be made without departing from the spirit or scope of the claims.

What is claimed is:

1. A method of quantifying microvascular function in a patient, the method comprising:
stabilizing a test portion of the patient to analyze;
measuring a microvascular blood flow parameter of the test portion using a first spectral imaging technique;
measuring a microvascular reserve parameter of the test portion using a second spectral imaging technique;
measuring a tissue respiration parameter of the test portion using a third spectral imaging technique;
measuring a microvascular permeability parameter of the test portion using a fourth spectral imaging technique; and
processing the microvascular blood flow parameter, the microvascular reserve parameter, the tissue respiration parameter, and the microvascular permeability parameter together using a processor configured to generate a summary microvascular parameter corresponding to the microvascular function in the patient.

2. The method of claim 1, wherein the first, second, third, and fourth spectral imaging techniques each comprise microscopic spectral imaging, endoscopic spectral imaging, camera spectral imaging, or a combination thereof.

3. The method of claim 1, wherein the first, second, third, and fourth spectral imaging techniques each comprise a multispectral or hyperspectral imaging technique.

4. The method of claim 1, wherein the microvascular blood flow parameter, the microvascular reserve parameter, the tissue respiration parameter, and the microvascular permeability parameter are each measured in the epidermis, dermis, hypodermis, buccal mucosa, palpebral inferior region, palpebral superior region, auricle, and/or outer surface of any internal organ during a surgical, laparoscopic, or endoscopic procedure.

5. The method of claim 1, wherein the first spectral imaging technique comprises a line scan spectroscopic speckle technique.

6. The method of claim 1, wherein the first spectral imaging technique comprises diffuse correlation spectroscopy, diffuse optical spectroscopy, or a combination thereof.

7. The method of claim 1, wherein the first spectral imaging technique uses a wavelength from about 400 nm to about 3000 nm.

8. The method of claim 1, wherein the second spectral imaging technique is configured to observe capillary response to an ischemic stimulus where a blood supply is at least temporarily changed or shut off.

9. The method of claim 1, wherein the second spectral imaging technique uses a first wavelength from about 530 nm to about 580 nm and a second wavelength from about 440 nm to about 460 nm.

10. The method of claim 1, wherein the third spectral imaging technique comprises pulse oximetry to image hemoglobin (Hb) and oxyhemoglobin (HbO₂) molecules.

11. The method of claim 1, wherein the third spectral imaging technique uses a first wavelength from about 530 nm to about 580 nm and a second wavelength from about 440 nm to about 460 nm.

12. The method of claim 1, wherein the fourth spectral imaging technique is configured to image water (H₂O) permeating a plurality of vascular walls.

13. The method of claim 1, wherein the fourth spectral imaging technique uses one or more wavelength bands comprising 2900 nm, 1950 nm, 1450 nm, 1200 nm, 900 nm, 820 nm, and 730 nm.

14. A method of quantifying microvascular function in a patient, the method comprising:
measuring two or more microvascular parameters selected from the group consisting of a microvascular blood flow parameter, a microvascular reserve parameter, a tissue respiration parameter, and a microvascular permeability parameter; and
processing the two or more microvascular parameters using a controller configured to generate a summary microvascular parameter corresponding to the microvascular function in the patient;
wherein the two or more microvascular parameters are measured using a spectral imaging technique comprising microscopic spectral imaging, endoscopic spectral imaging, camera spectral imaging, or a combination thereof.

15. The method of claim 14, wherein the two or more microvasculature parameters include the microvacular blood flow parameter and the microvascular blood flow parameter is quantified with a line scan spectroscopic speckle technique using a wavelength from about 530 nm to about 580 nm.

16. The method of claim 14, wherein the two or more microvasculature parameters include the microvascular reserve parameter and the microvascular reserve parameter is quantified with the spectral imaging technique by observing capillary response to an ischemic stimulus where the blood supply is at least temporarily changed or shut off; and
wherein the spectral imaging technique uses a first wavelength from about 530 nm to about 580 nm and a second wavelength from about 440 nm to about 460 nm.

17. The method of claim 14, wherein the two or more microvasculature parameters include the tissue respiration parameter and the tissue respiration parameter is quantified with the spectral imaging technique by using pulse oximetry to image hemoglobin (Hb) and oxyhemoglobin (O₂Hb) molecules; and
wherein the spectral imaging technique uses a first wavelength from about 530 nm to about 580 nm and a second wavelength from about 440 nm to about 460 nm.

18. The method of claim 14, wherein the two or more microvasculature parameters include the microvascular permeability parameter and the microvascular permeability parameter is quantified with the spectral imaging technique by imaging water (H₂O) permeating through a plurality of vascular walls; and
wherein the spectral imaging technique uses one or more wavelength bands comprising 2900 nm, 1950 nm, 1450 nm, 1200 nm, 900 nm, 820 nm, and 730 nm.

19. The method of claim 14, wherein the two or more microvascular parameters are measured by detecting carbon dioxide, oxygen, hemoglobin, carboxyhemoglobin, deoxyhemoglobin, methemoglobin, nitric oxide, or a combination thereof.

20. An instrument used to quantify microvascular function comprising:
a spectral imaging device configured to measure two or more microvascular parameters selected from the group consisting of a microvascular blood flow parameter; a microvascular reserve parameter; a tissue respiration parameter; and a microvascular permeability parameter; and
a processor configured to generate a summary microvascular parameter from the two or more microvascular parameters.
